(19) United States
(12) Patent Application Publication
(10) Pub. No.: US 2005/0032898 A1
(43) Pub. Date: Feb. 10, 2005

(54) Oral amino acid composition

(75) Inventor: Masaru Ohtani, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/885,744

(22) Filed: Jul. 8, 2004

(30) Foreign Application Priority Data
Jul. 8, 2003 (JP) 2003-193737

(51) Int. Cl. A61K 31/198

(52) U.S. Cl. 514/561; 514/563; 514/565

(57) ABSTRACT

Amino acid compositions which contain 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine may be taken orally and are effective for reducing body fat without dietary restrictions and exercise.

### CROSS REFERENCES TO RELATED APPLICATIONS

This application claims priority to Japanese Patent Application No. 193737/2003, filed on Jul. 8, 2003, and which is incorporated herein by reference in its entirety.

### BACKGROUND OF THE INVENTION

1. Field of the Invention

The present invention relates to diet compositions which contain amino acids as active components. The compositions may be taken orally and show the effects of increasing the amount of muscle and reducing the amount of body fat. The present invention also relates to methods for increasing muscle mass and methods for reducing body fat by administering such a composition to a subject in need thereof.

2. Discussion of the Background

In the past, diets in which the amount and type of meal(s) are restricted have been used to maintain and enhance beauty and health. Though various methods of reducing body weight or body fat by the use of diet products (nutritional supplements) without meal restriction have recently been studied, a sufficiently effective diet product has not been achieved yet at present.

In the meanwhile, in recent years, various knowledge has been accumulated on the physiological functions of amino acid compositions. For example, it has been found that compositions containing five types of amino acids, L-arginine, L-glutamine, L-valine, L-isoleucine, and L-leucine, exhibit a hematopoiesis function and a nutrient state improving function (see, JP-A-2002-3372). Further, compositions containing L-lysine, methionine, L-threonine, L-histidine, L-proline, L-phenylalanine, and L-tryptophan in addition to the above-mentioned five types of amino acids exhibit the effect of relieving muscle fatigue and mental fatigue occurring along therewith such as a feeling of lassitude (see, JP-A-8-198748). However, it has not been known whether such amino acid compositions show the effects of reducing body weight or body fat.

Thus, there remains a need for compositions which are effective for reducing body weight and reducing body fat. There also remains a need for compositions which are effective for increasing muscle mass. There also remains a need for methods for reducing body weight and reducing body fat and methods for increasing muscle mass.

### SUMMARY OF THE INVENTION

Accordingly, it is one object of the present invention to provide novel amino acid-containing diet products.

It is another object of the present invention to provide novel amino acid compositions which are effective for reducing the amount of body fat.

It is another object of the present invention to provide novel amino acid compositions which are effective for increasing the amount of muscle mass. It is another object of the present invention to provide novel amino acid compositions which may be consumed orally.

It is another object of the present invention to provide novel methods for reducing body weight and reducing body fat.

It is another object of the present invention to provide novel methods for increasing muscle mass.

These and other objects, which will become apparent during the following detailed description, have been achieved by the inventor's discovery that body fat can be reduced while increasing the amount of muscle by taking a composition containing five types of amino acids, L-arginine, L-glutamine, L-valine, L-isoleucine, and L-leucine. The present invention has thus been accomplished by this finding.

Accordingly, the present invention provides the following embodiments:
(1) An amino acid composition, consisting essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine.

(2) A method for reducing body fat, comprising administering an effective amount of an amino acid composition comprising 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine to a subject in need thereof.

(3) A method for increasing the amount of muscle mass, comprising administering an effective amount of an amino acid composition comprising 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine to a subject in need thereof.

The amino acid compositions of the present invention may be given orally and show the desired effects. The daily intake of the composition is suitably 3 to 15 g, more preferably 6 to 10 g. Moreover, the desired effects can be obtained by taking the composition without particular exercise and dietary restrictions.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a diagram showing the changes of the body weight, the body fat, and the waist, measured before (pre) and after (post) the intake of the amino acid composition in Example 1. From left to right, the first pair of bars reflect body weight (kg); the second pair of bars reflect lean body weight (kg); the third pair of bars reflect body fat weight (kg); the fourth pair of bars reflect percent body fat (%); and the fifth pair of bars reflect waist size (cm).

### DETAILED DESCRIPTION OF THE PREFERRED EMBODIMENTS

Thus, in a first embodiment, the present invention provides novel compositions which consist essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine.

The various embodiments of the present invention will now be described below.

The amino acid composition of the present invention may be prepared by simply mixing the above-mentioned five types of amino acids in the above-mentioned predetermined ratios, or by any common method using an appropriate additive. Examples of such additives include those for controlling or improving the taste, such as fruit juices, dextrins, cyclic oligosaccharides, saccharides (fructose, glucose, liquid sugars, sucrose, etc.), acidulants, flavorings, and green powdered teas; those for improving the texture, such as emulsifiers, collagens, whole milk powders, polysaccharide thickeners, and agars for jelly drinks; and those which are commonly used as components of health foods, such as amino acids other than the above-mentioned five types of amino acids, vitamins, egg shell calcium, calcium pantothenate, other minerals, royal jellies, propolis, honeys, food fibers, agaricus, chitin, chitosan, Chinese herbal medicines, chondroitin, and capsaicin.

The product of the amino acid composition of the present invention may be in any form without particular restrictions as long as the composition can be consumed orally (eaten). The composition may be in the form of a powder, granule, tablet, liquid (drink, jelly drink, etc.), candy (chocolate, etc.), etc. using an appropriate vehicle. In an extreme case, the composition may be a simple particle mixture (powder) of the above-mentioned five types of amino acids in the above mixing ratio. The powder of the composition may be taken directly with water like powdered medicines. Further, the powder of the composition may be taken such that the powder is wrapped in a wafer paper or dissolved in water, milk, juice, etc.

In view of the results of Example 1, described below, the amino acid composition of the present invention shows remarkable effects for reducing body fat, increasing the amount of muscle, etc. when the total intake of the essential components of the 5 amino acids with the above ratio is 7 g or more per day. On the other hand, an excess amount of the amino acids cannot further improve the effects, and the maximum intake of the amino acids may be approximately 15 g per day. The preferred daily intake within the range is, for example, approximately 6 to 10 g/day. Thus, the amino acid composition of the present invention preferably contains the amino acids essential components, in an amount such that the above intake condition is satisfied, depending also on the number of times the composition is to be taken per day.

In another embodiment, the present invention provides novel methods for reducing body fat and increasing muscle mass, by administering an effective amount of a composition comprising 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

In a preferred embodiment, the subject to whom the composition is taken by or administered to is a human, including women, men, adult humans, and children.

In another preferred embodiment, the composition is administered to or taken by the subject daily. The composition is preferably administered or consumed daily over a time period of from 1 day until the desired effect is achieved. Thus, the composition may be administered or consumed over a time period ranging from days, to weeks, to months, and even years. In the context of the present invention it is to be understood that the term “administering” includes and encompasses those situations in which the subject orally consumes the compositions without the intervention of another individual.

In another preferred embodiment, the present invention provides a method of reducing body fat, comprising administering an effective amount of a composition consisting essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

In another preferred embodiment, the present invention provides a method of increasing muscle mass, comprising administering an effective amount of a composition consisting essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

Other features of the invention will become apparent in the course of the following descriptions of exemplary embodiments which are given for illustration of the invention and are not intended to be limiting thereof.

### EXAMPLES

### Example 1

The effects of amino acid intake on six (6) test subjects (women, aged 42.0+3.6 years old, height 156.9±2.2 cm) were evaluated using the indexes of body weight, body fat, etc. 7.3 Gram per day of an amino acid composition containing L-arginine, L-glutamine, L-valine, L-isoleucine, and L-leucine were taken orally by each test subject for two weeks, and the body weight, the lean body weight, the body fat weight, the body fat percentage, and the waist of each subject were measured before and after the intake, there being no dietary restrictions. The amino acid composition contained 1.99 g of L-arginine, 1.36 g of L-glutamine, 1.09 g of L-valine, 1.17 g of L-isoleucine, and 1.67 g of L-leucine and was given each day.

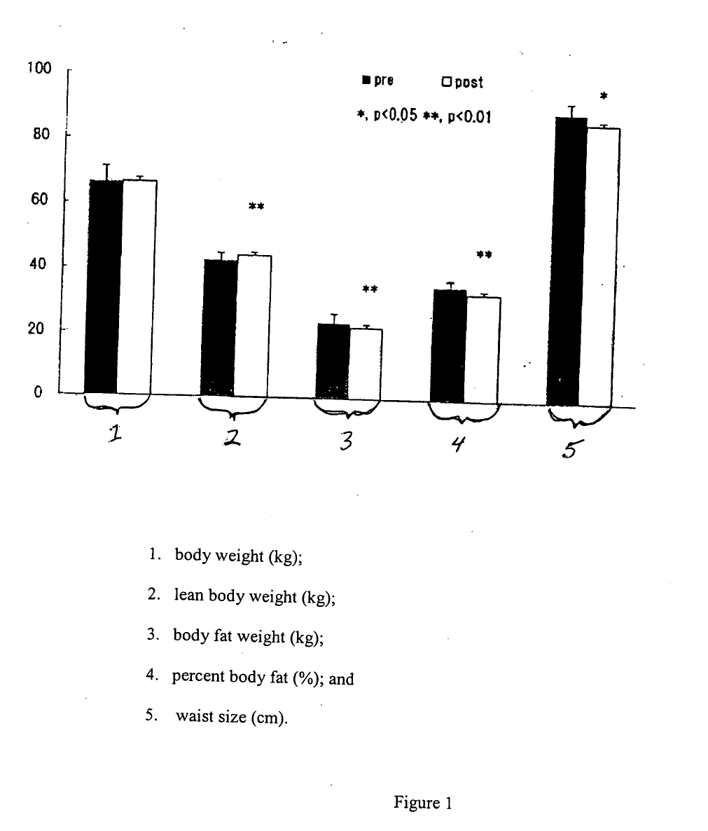

The results are shown in Table 1, below, and in FIG. 1. As shown in Table 1 and FIG. 1, by the amino acid composition intake, the amount of muscle was increased by approximately 1.6 kg, and the body fat weight was decreased by approximately 1.5 kg. The waist was also reduced by approximately 4 cm, and thus it was clear that the proportions of the test subjects were improved.

TABLE 1

| | Average | | | SEM | | |
|---|---|---|---|---|---|---|
| | Before | After | | Ratio of | Before | After |
| | intake | intake | | difference | intake | intake |
| | (pre) | (post) | Difference | (%) | (pre) | (post) |
| Body weight (kg) | 66.5 | 66.7 | 0.12 | 0.17 | 4.58 | 4.6 |
| Lean body weight (kg) | 42.7 | 44.2 | 1.57 | 3.67 | 2.25 | 2.29 |
| Body fat weight (kg) | 23.9 | 22.4 | −1.45 | −6.08 | 2.79 | 2.73 |
| Body fat percentage (%) | 35.4 | 33.2 | −2.20 | −6.21 | 1.9 | 2 |
| Waist (cm) | 89.7 | 86.4 | −3.28 | −3.66 | 3.07 | 3.26 |

As described above, it is clear that the amino acid composition of the present invention can reduce body weight and body fat to be effective for diet. Further, the amino acid composition uses the amino acids as the active components, thereby having an advantage in safeness.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition, consisting essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine.

2. The composition of claim 1, which is in a form suitable for oral consumption.

3. The composition of claim 1, which consists of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine.

4. A method of reducing body fat, comprising administering an effective amount of a composition comprising 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

5. The method of claim 4, wherein said composition is administered orally.

6. A method of reducing body fat, comprising administering an effective amount of a composition consisting essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

7. The method of claim 6, wherein said composition is administered in an amount sufficient to deliver said L-arginine, L-glutamine, L-valine, L-isoleucine, and L-leucine to said subject in a total amount of 3 to 15 grams per day.

8. The method of claim 6, wherein said composition is administered in an amount sufficient to deliver said L-arginine, L-glutamine, L-valine, L-isoleucine, and L-leucine to said subject in a total amount of 6 to 10 grams per day.

9. The method of claim 6, wherein said subject is a human.

10. The method of claim 6, wherein said subject is a woman.

11. The method of claim 6, wherein said subject is a man.

12. The method of claim 6, wherein said subject is an adult human.

13. The method of claim 6, wherein said composition is administered to said subject daily.

14. The method of claim 6, wherein said composition is administered orally.

15. A method of increasing muscle mass, comprising administering an effective amount of a composition comprising 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

16. The method of claim 15, wherein said composition is administered orally.

17. A method of increasing muscle mass, comprising administering an effective amount of a composition consisting essentially of 10 to 40 parts by weight of L-arginine, 10 to 40 parts by weight of L-glutamine, 5 to 20 parts by weight of L-valine, 8 to 30 parts by weight of L-isoleucine, and 10 to 35 parts by weight of L-leucine, to a subject in need thereof.

18. The method of claim 17, wherein said composition is administered in an amount sufficient to deliver said L-arginine, L-glutamine, L-valine, L-isoleucine, and L-leucine to said subject in a total amount of 3 to 15 grams per day.

19. The method of claim 17, wherein said composition is administered in an amount sufficient to deliver said L-arginine, L-glutamine, L-valine, L-isoleucine, and L-leucine to said subject in a total amount of 6 to 10 grams per day.

20. The method of claim 17, wherein said subject is a human.

21. The method of claim 17, wherein said subject is a woman.

22. The method of claim 17, wherein said subject is a man.

23. The method of claim 17, wherein said subject is an adult human.

24. The method of claim 17, wherein said composition is administered to said subject daily.

25. The method of claim 17, wherein said composition is administered orally.
